(19) United States
(12) Patent Application Publication
(10) Pub. No.: US 2021/0330515 A1
(43) Pub. Date: Oct. 28, 2021

(54) EARWAX REMOVAL DEVICE

(71) Applicant: Kwang Su KIM, Seongnam-si (KR)

(72) Inventor: Kwang Su KIM, Seongnam-si (KR)

(21) Appl. No.: 17/203,507

(22) Filed: Mar. 16, 2021

(30) Foreign Application Priority Data
Apr. 23, 2020 (KR) 10-2020-0049245

(51) Int. Cl. A61F 13/38

(52) U.S. Cl. CPC A61F 13/38

(57) ABSTRACT

The present invention provides an earwax removal device comprising: a housing including an interior space, a body part having a rod shape and received into the interior space; a wing part including at least one hingeable wing and connecting to one end of the body part, and a pressurized module moving the body part forward and backward; wherein when the body part is moved forward, and the one end of the body part is exposed to outside of the housing, the wing part is spread; and when the body part is moved backward, and the one end of the body part is received inside the interior space, the wing part is folded.

### FIELD OF THE INVENTION

The present invention relates to an earwax removal device and, more particularly, an earwax removal device to remove various types of earwax, such as dry, wet, and oily earwax, safely and effectively.

### BACKGROUND OF THE INVENTION

Conventional types of earwax removal devices are an ear swab, a cotton swab, or other similar tools. Elderly and children who have poor manipulation skills of tools, may fail delicate control of the device. They may insert the tool too deep into the ear canal, which results in damage or perforation of tympanic membrane. Furthermore, when they overly force an ear swab or a cotton swab into skin surface, it may cause skin wounds or inflammation. In addition, when people rub their inner ear to remove earwax, it causes earwax flakes.

According to prior Korean Patent Publication No. 10-2016-0070586 to solve these problems, it discloses an inner ear cleaner, which sucks in and pulls out earwax through a suction part and. As illustrated in FIG. 1, the inner ear cleaner comprises: a cylinder including a motor 20′ and a pan 22′ which connect each other, vents 12′ disposed in the middle, a body 10′ which a filter 26′ is inserted into the inner side of the front thereof and a battery 24′ is inserted into the rear thereof, a handle 14′ which includes an operating switch 16′, and a suction pipe 30′ that connects to the front of the body 10′ with screws, that includes an interior space part 34′, and that has a shape with narrow ends toward the front thereof. The suction pipe 30′ includes bumps 32′ at the end of the outer side, a bluetooth 40′, and a lamp 41′ for brightening.

However, according to the inner ear cleaner, it requires high maintenance costs caused by batteries for electricity supply. The complexity of the structure also makes its manufacturing process complicated. In addition, when the suction pipe sucks in earwax, excessive suction force may cause damage to the eardrum.

The present invention provides an earwax removal device which can rake out earwax quick and simple without excessive force and be stored in a sanitary way without damage of the device. In addition, the present invention provides an earwax removal device having a simple structure so that the production time and unit price can be reduced.

### SUMMARY OF THE INVENTION

The present invention provides an earwax removal device comprising: a housing including an interior space, a body part having a rod shape and received into the interior space; a wing part including at least one hingeable wing and connecting to one end of the body part, and a pressurized module moving the body part forward and backward; wherein when the body part is moved forward, and the one end of the body part is exposed to outside of the housing, the wing part is spread; and when the body part is moved backward, and the one end of the body part is received inside the interior space, the wing part is folded; wherein the body part has a cylindrical shape and comprises a front side, a back side, and a lateral side connecting the front and the back side; wherein the pressurized module comprises: a screw axis part having a helical groove formed in an outer circumference side of the other end of the body part; a rotation member that rotatably connects to an inner side of the housing and that has a screw thread on an inner side thereof, the screw thread forming a screw joint with the screw axis part; a rotation supporting part having a ring shape and supporting the rotation member to be rotatable inside the housing, and an insertion part that is disposed in front of the rotation supporting part and that has an inner shape corresponding to the body part to receive the body part; wherein the rotation member comprises: a first joint part that rotatably connects to the inner side of the rotation supporting part of the housing; a second joint part that extends from one end of the first joint part toward a front of the housing with a smaller diameter than that of the first joint part and that has the screw thread formed on an inside thereof, and a rotation control part that is connected to the other end of the first joint part and that is disposed at a rear of the housing; wherein the body part and the rotation member form the screw joint, when the rotation member is rotated clockwise or counterclockwise, and the body part moves forward to the front of the housing, the wing part that has been received inside the housing, is spread, and when the rotation member is rotated counterclockwise or clockwise, and the body part goes backward, the wing part that has been spread, is folded and received into the housing.

The housing is a pipe shape having openings in a front and a rear whereof, and a length of the housing is shorter than that of the body part; wherein at least one concave groove is formed in an inner side of the housing, and at least one raised sill having a shape corresponding to the concave groove is formed on the outer circumference side of the body part.

The end of a wing in the wing part is covered with cotton.

### BRIEF DESCRIPTION OF THE DRAWINGS

FIG. 1 illustrates an inner ear cleaner of prior art.

FIGS. 2A and 2B are a schematic side view showing an earwax removal device of the present invention.

FIG. 3A is an exploded perspective view showing a pressurized module according to a first embodiment of the present invention.

FIG. 3B is a sectional view showing a pressurized module according to the first embodiment of the present invention.

FIG. 4 is a drawing showing when the earwax removal device is in use according to the first embodiment of the present invention.

FIG. 5 is a sectional view showing a pressurized module according to a second embodiment of the present invention.

FIG. 6 is a side view showing a pressurized module according to another embodiment of the present invention.

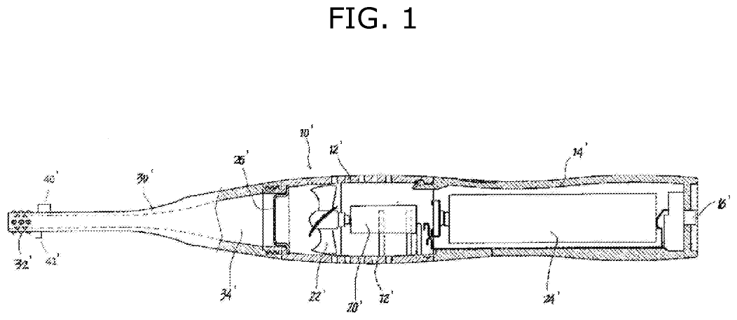

### DETAILED DESCRIPTION EMBODIMENTS OF THE INVENTIONS

Hereafter, the embodiments of the present invention will be described in detail with reference to the accompanying drawings. In the drawings, the thickness of lines or the sizes of the elements may be exaggerated for clarity.

The terminology used herein is defined for the purpose of describing functions of the present invention; thus, it may be varied depending on a user intent or a convention in this technical field.

The embodiments below are not restricting or limiting the scopes of the inventive concepts but illustrating elements of the claims. The embodiment that includes the equivalent of the element of the following claim shall be within the scope of the present invention.

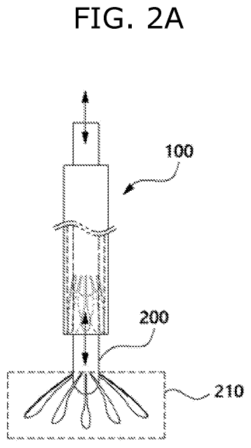

FIGS. 2A and 2B are schematic side views showing an earwax removal device of the present invention.

Referring to FIG. 2A, the earwax removal device comprises a housing 100 including an interior space, a body part 200 having a rod shape and received into the interior space, and a wing part 210 including at least one wing. When the body part 200 is moved forward, an end of the body part 200 is exposed to outside, and at the same time, the wing part 210 is spread; when the body part 200 is moved backward, the end of the body part 200 is received into the interior space of the housing 100, and at the same time, the wing part 210 is folded. The body part 200 has a shape extending along longitudinal direction of the side of the housing 100. It is desired that the external diameter of the body part 200 is smaller than the internal diameter of the housing 100. The wing part 210 is preferably made of elastic materials.

Referring to FIG. 2B based on the basic structure of FIG. 2A, a first embodiment of the present invention is explained as follows. The body part 200 comprises the front face 201 that has a cylinder shape and opens in the front, the rear face 202 that is disposed in the rear opposite of the front, and the side face 203 that connects between the front face 201 and the rear face 202. In addition, the body part 200 further comprises a pressurized module 300 that moves the body part 200 forward and backward. When the body part 200 is moved forward using the pressurized module 300, an end of the body part 200 is exposed to outside, and at the same time, the wing part 210 is spread. When the body part 200 is moved backward using the pressurized module 300, the end of the body part 200 is received into the interior space of the housing 100, and at the same time, the wing part 210 is folded. According to the structure described above, the earwax removal device of the present invention is easy to use because the wing part 210 is spread and folded depending on shifting the body part 100 back and forth. In addition, the earwax removal device has a simple structure, which allows the production time and unit price to be reduced. Furthermore, the earwax removal device can rake out earwax quick and simple without excessive force using the wing part 210 in a state of spreading. The wing part 210 in a state of spreading can prevent the body part 200 from being inserted too far into the ear canal.

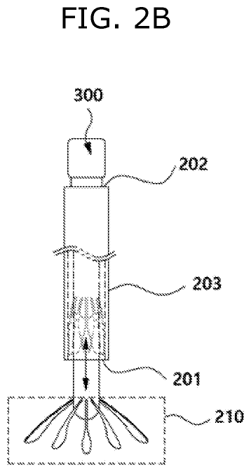

FIG. 3A is an exploded perspective view showing a pressurized module according to a first embodiment of the present invention. FIG. 3B is a sectional view showing a pressurized module according to the first embodiment of the present invention.

Referring to FIG. 3A, a pressurized module 300 comprises: a screw axis part 310 that has a helical groove 311 formed in the outer circumference side of the other end of the body part 200; a rotation member 320 that rotatably connects to the inner side of the housing 100 and has a screw thread 321 in an inner side thereof, the screw thread forming a screw joint with the screw axis part 310; a rotation supporting part 330 that has a cylindrical circumference shape in an inner side of the housing 100 and supports the rotation member 320 to rotate; and an insertion part 340 that is disposed in front of the rotation supporting part 330, receives the body part 200, and has the inner shape corresponding to the shape of the outer shape of the body part 200 to receive the body part 200. In addition, the rotation member 320 comprises: a first joint part 320a that rotatably connects to the inner side of the rotation supporting part 330 of the housing 100; a second joint part 320b that extends from one end of the first joint part 320a toward the front of the housing 100 with a smaller diameter than that of the first joint part 320a, and has the screw thread formed on the inside; and a rotation control part 320c that is connected to the other end of the first joint part 320a and disposed at the rear of the housing 100.

Referring to FIG. 3B, an earwax removal device may further comprise an auxiliary rotation member 10. The auxiliary rotation member 10 may be disposed between the rotation supporting part 330 of the housing 100 and the first joint part 320a of the rotation member 320 to help rotational motion of the housing 100 and the rotation member 320. The auxiliary rotation member 10 may be a bearing or a ring-shape packing with high lubricity property. Furthermore, the auxiliary rotation member 10 may be covered with an additional lubricant.

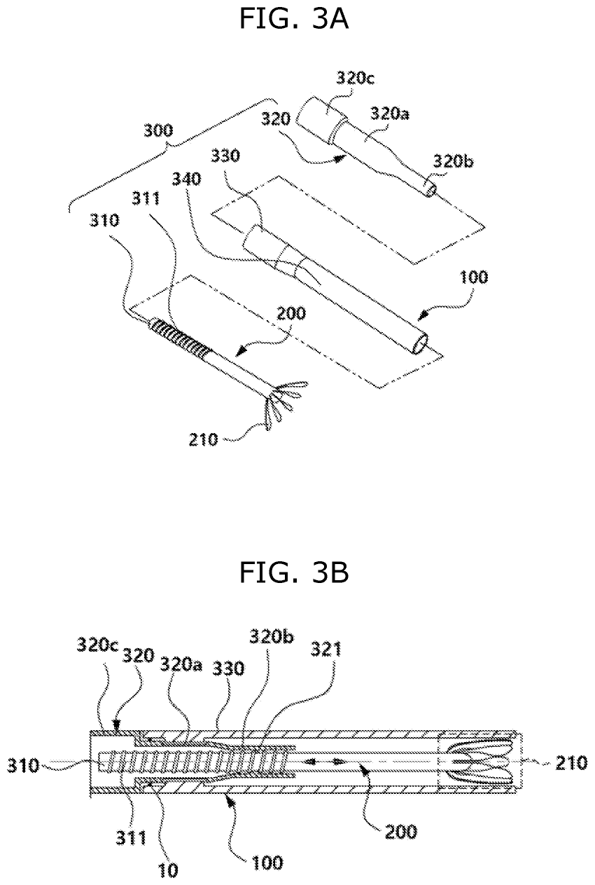

FIG. 4 is a drawing showing when the earwax removal device is in use according to the first embodiment of the present invention.

Referring to FIG. 4, the earwax removal device according to the first embodiment of the present invention is operated as follows. When the rotation member 320 is rotated clockwise or counterclockwise, the body part 200 moves forward to the front of the housing 100. This motion enables the wing part 210, which has been folded and received inside the housing 100, to be exposed and spread. Then, a user can easily rake out earwax using the wing part 210 spread. When not in use, the rotation member 320 can be rotated counterclockwise or clockwise, which makes the body part 200 goes backward. This enables the wing part 210, which has been spread, to be folded and received into the housing 100.

According to the structure described above, the end portion of the body part 200, which is inserted into the ear canal, can be stored inside the housing 100. Therefore, the earwax removal device can be kept in a sanitary way without damage of the wing part 210 and be easily portable.

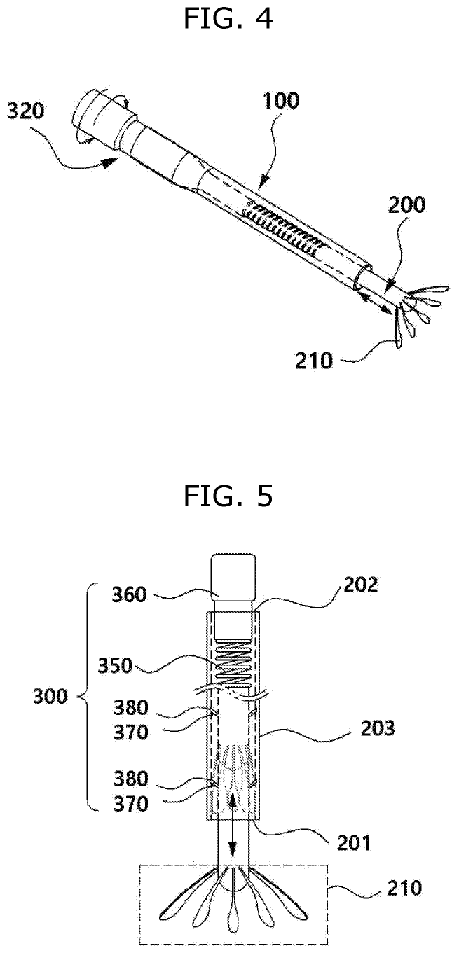

FIG. 5 is a sectional view showing a pressurized module according to a second embodiment of the present invention.

The pressurized module 300 is received inside the housing 10. The pressurized module 300 comprises: an elastic spring 350 one end of which meets one side of the body part 200; two raised sills 370 that protrude from the outer circumstance side of the body part 200 and are separately disposed along the longitudinal direction of the body part 200; and two concave grooves 380 that have a shape corresponding to the shape of the raised sill 370 and are disposed along the longitudinal direction of the housing 100.

The back side of the raised sill 370 has an inclined plane with a certain angle. When the pressurized module 360 pressurizes the elastic spring 350, the body part 200 moves forward by elastic force of the elastic spring 350. In addition, the raised sill 370 is inserted into the concave groove 380, which makes the body part 200 fixed at a position after moving forward. When the pressurized module 360 repressurizes the elastic spring 350, the raised sill 370 gets out of the concave groove 380, and the body part 200 moves backward by elastic restoring force.

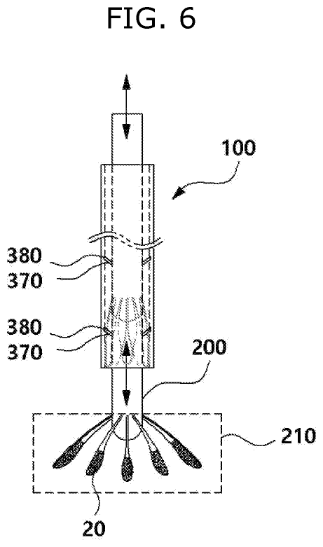

FIG. 6 is a side view showing pressurized module 300 according to another embodiment of the present invention.

Referring to FIG. 6, the housing 100 is a pipe shape having openings in the front and the rear. The length of the housing 100 may be shorter than that of the body part 200.

In addition, at least one concave groove 380 is formed in the inner side of the housing 100. At least one raised sill 370 having a shape corresponding to the concave groove 380, is formed on the outer circumference side of the body part 200. The concave groove 380 and the concave groove 380 enables the body part 200 to be fixed at a position after moving forward.

Furthermore, an end of a wing in the wing part 210 may be covered with cotton 20, or cotton swabs, which prevents skin surface of the inner ears from getting wounds or inflammations.

While embodiments of the present invention have been described, the present invention is not limited to what has been particularly shown. It would be apparent that many more modifications and variations than mentioned above are possible by an ordinary person skilled in the art. The scope of the present invention includes scopes of appended claims, modifications, and variations.

What is claimed is:

1. An earwax removal device comprising:
a housing including an interior space,
a body part having a rod shape and received into the interior space;
a wing part including at least one hingeable wing and connecting to one end of the body part, and
a pressurized module moving the body part forward and backward;
wherein when the body part is moved forward, and the one end of the body part is exposed to outside of the housing, the wing part is spread; and when the body part is moved backward, and the one end of the body part is received inside the interior space, the wing part is folded;
wherein the body part has a cylindrical shape and comprises a front side, a back side, and a lateral side connecting the front and the back side;
wherein the pressurized module comprises:
a screw axis part having a helical groove formed in an outer circumference side of the other end of the body part;
a rotation member that rotatably connects to an inner side of the housing and that has a screw thread on an inner side thereof, the screw thread forming a screw joint with the screw axis part;
a rotation supporting part having a ring shape and supporting the rotation member to be rotatable inside the housing, and
an insertion part that is disposed in front of the rotation supporting part and that has an inner shape corresponding to the body part to receive the body part;
wherein the rotation member comprises:
a first joint part that rotatably connects to the inner side of the rotation supporting part of the housing;
a second joint part that extends from one end of the first joint part toward a front of the housing with a smaller diameter than that of the first joint part and that has the screw thread formed on an inside thereof, and
a rotation control part that is connected to the other end of the first joint part and that is disposed at a rear of the housing;
wherein the body part and the rotation member form the screw joint,
when the rotation member is rotated clockwise or counterclockwise, and the body part moves forward to the front of the housing, the wing part that has been received inside the housing, is spread, and
when the rotation member is rotated counterclockwise or clockwise, and the body part goes backward, the wing part that has been spread, is folded and received into the housing.

2. The earwax removal device according to claim 1,
wherein the housing is a pipe shape having openings in a front and a rear whereof, and a length of the housing is shorter than that of the body part;
wherein at least one concave groove is formed in an inner side of the housing, and at least one raised sill having a shape corresponding to the concave groove is formed on the outer circumference side of the body part.

3. The earwax removal device according to claim 2, an end of a wing in the wing part is covered with cotton.

4. The earwax removal device according to claim 1, an end of a wing in the wing part is covered with cotton.
